(19) United States
(12) Patent Application Publication
(10) Pub. No.: US 2011/0046632 A1
(43) Pub. Date: Feb. 24, 2011

(54) Medical Tool And Method For Forming Holes

(76) Inventor: Felix Quevedo, Palmetto Bay, FL (US)

Correspondence Address: MCHALE & SLAVIN, P.A., 2855 PGA BLVD, PALM BEACH GARDENS, FL, 33410, US

(21) Appl. No.: 12/792,505

(22) Filed: Jun. 2, 2010

Related U.S. Application Data
(60) Provisional application No. 61/183,177, filed on Jun. 2, 2009.

(51) Int. Cl. A61B 17/56; A61B 17/34

(52) U.S. Cl. 606/102; 606/185

(57) ABSTRACT

A medical tool and method are provided. The tool includes a shank for insertion into an animal to produce a hole, inject material and/or extract a sample. The shank is provided with color zones which, when referenced to a portion of a body part, will indicate depth of penetration of the shank.

### RELATED APPLICATION

This application claims the benefit under 35 U.S.C. 119(e) of U.S. Provisional Patent Application No. 61/183,177, filed Jun. 2, 2009, entitled, “Medical Tool And Method For Forming Holes”, the entirety of which is incorporated herein by reference.

### FIELD OF INVENTION

The invention relates to a medical tool for forming holes, particularly in bone and tissue for medical procedures including implanting, extracting samples, injections and surgery.

### BACKGROUND OF THE INVENTION

Tools for forming holes for sampling or surgical procedures are well known. Such tools can be found described in U.S. Pat. Nos. 3,478,435, 5,013,318, 5,361,766, 5,928,243 and 7,134,216, and in published US Patent Application 2007/0088366. A common feature of these tools is a depth indicating structure. All feature a scale with depth indicating lines and a reference line forming structure that, when lined up with a depth line, indicates depth of hole or penetration. Such devices include surgical devices for forming holes in bone such as pedicle probes and jam-shidi needles. While the devices are effective at forming holes and extracting samples or injecting fluids, the depth indicating structures pose problems.

The above patents typically disclose what appear to be ruled scales with numerals designated by lines which, when matched to an indicator, indicate a depth of penetration or a thickness of a bone or the like by passing through the bone. Many of these tools are relatively small and the scales are thus relatively small. Many medical procedures, especially when performed percutaneously, do not allow a doctor or nurse to view the tool straight on, making the scale difficult or impossible to view. To determine depth, the user would have to move to reorient their line of sight, e.g., by moving their head to view the scale and indicator from a position that may not be possible to attain depending on the procedure.

There is thus a need for an improved medical tool to provide for improved accuracy and/or ability to use to measure depth of hole or penetration.

### SUMMARY

The present invention involves the provision of a medical tool with an improved depth indicating structure. The tool includes a probe with a shank portion having a distal end and a proximal end. The shank includes a longitudinally extending outer surface with a plurality of depth indicating zones along the length thereof. Immediately adjacent zones have different colors with each color zone representing a different distance range from the distal end of the shank. A handle member is mounted to the probe adjacent its proximal end so that a user can manipulate and use the tool.

The present invention also involves the provision of a method of determining depth of penetration of a medical tool into a portion of a patient for forming a hole in a surgical procedure, extracting a sample or injecting material into a patient. The method includes selecting a probe suitable for the particular medical procedure to be performed. Such probes include, but are not limited to, pedicle probes, jam-shidi needles, syringe needles and the like. The probe has a shank with a plurality of different color zones along at least a portion of the length of the shank. The shank is inserted into an animal bone, tissue or organ to a desired depth. A color is identified from a reference chart in which color represents a depth of penetration of the shank from its distal end. The depth of penetration may be determined before, during or after the procedure, depending on the specific procedure performed.

Other objects and advantages of the present invention will become apparent from the following detailed description of the invention.

### DESCRIPTION OF THE DRAWINGS

FIG. 1 is a side elevation view of one embodiment of the present invention in the form of a jam-shidi needle.

FIG. 2 is an enlarged fragmentary sectional side elevation of a shank portion of the Jam Shidi needle and a wire used with the needle, some dimensions being shown exaggerated for clarity.

FIG. 3 illustrates a depth/color reference chart.

FIG. 4 is a side elevation view of an attachment for use with a probe as shown in FIG. 1.

FIG. 5 is a side elevation view of an alternate embodiment of the probe shown in FIG. 1.

Like numbers used throughout the drawings designate like or similar parts and/or construction.

### DETAILED DESCRIPTION

While the present invention is susceptible of embodiment in various forms, there is shown in the drawings and will hereinafter be described presently preferred embodiments with the understanding that the present disclosure is to be considered an exemplification of the invention and is not intended to limit the invention to the specific embodiments illustrated.

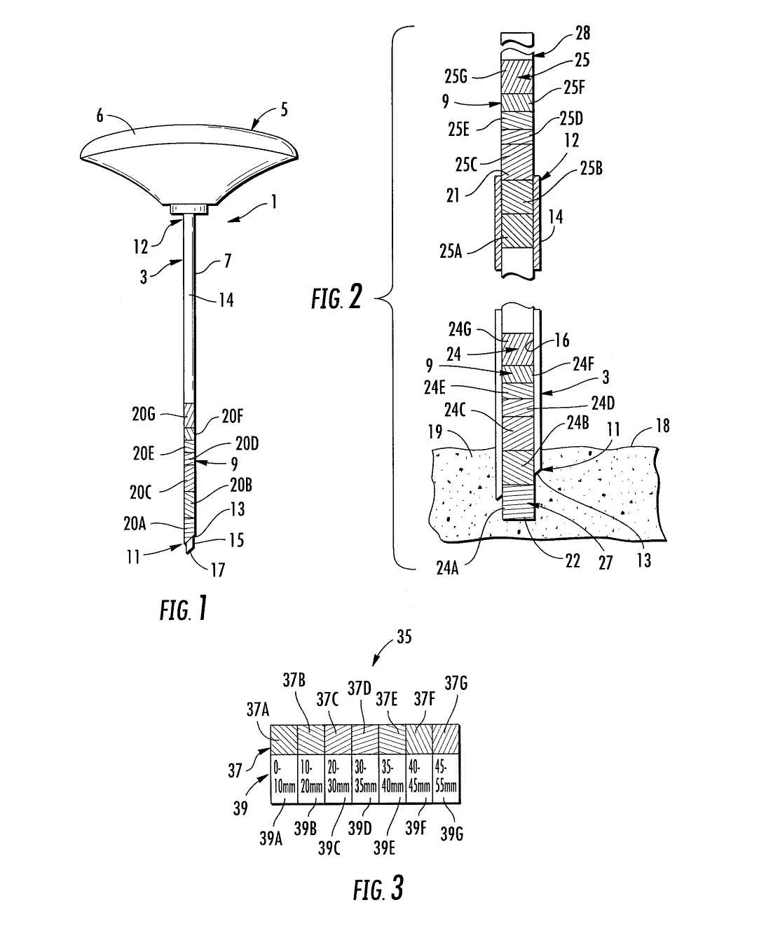

FIG. 1 shows one embodiment of the invention in the form of a jam-shidi needle. However, it is to be understood that the invention may be embodied in other forms useful in medical and surgical procedures such as a trocar also useful in forming a hole in bone. Medical devices of this general type and their uses are well known in the art. They may be used to treat a patient such as a human or other animal. The reference numeral 1 designates generally a medical device having a probe portion 3 and a handle portion 5. The probe portion 3 includes a shank 7 adapted for use to form a hole in a portion, such as a bone of a patient or to be inserted into a preformed hole. The shank 7 is provided with a color coded depth scale designated generally 9.

The device 1 includes a shank 7 that has a distal end portion 11 and a proximal end portion 12. The shank has an outer surface 14 extending between the end portions 11, 12. The shank 7 can be any suitable shape and is preferably generally round in transverse cross section to allow it to be rotated during use, particularly when penetrating bone. As shown for the device 1, the shank 7 may be generally cylindrical or tapered, e.g., conical with increasing size from the distal end portion 11 toward the proximal end portion 12. The distal end portion 11 may be pointed as by beveling at 13 to provide a sharp means to easily form a hole by piercing a body portion through the application of generally linear movement of the device 1 under force applied by a user, such as a doctor or nurse. The force may be applied by a hammer type device if desired. The device 1 can be used to pierce tissue and bone. Such devices are commonly used to pierce bone, such as a pedicle for spinal surgery. When used to pierce bone, it is preferred that the shank be a hardened metal alloy like stainless steel or titanium. To improve resistance to bending, the shank is hollow whereby a rod 15 can be removably positioned through a central bore 16 (FIG. 2) and can be provided with a sharp distal end portion 17. Preferably, the rod 15 and the bore 16 have generally round cross sectional shapes. The rod 15 may be fixed against longitudinal movement relative to the shank 7 during hole forming. The rod 15 may be a trocar if desired.

The device 1 includes a handle 5 mounted to the probe portion 3. The handle 5 may be of any suitable type such as the illustrated T-shaped handle, an L-shaped handle or even a straight handle depending on the use the device is designed for or user preference. The handle 5 may be secured to the probe portion 3 in any suitable manner such as by threaded interengagement or clamping as with jaws. Preferably, the handle 5, or a portion of the handle 5, such as the top portion 6, is removable to provide access to the proximal end 21 of the through bore 16 for a purpose described below.

The device 1 is provided with a depth scale 9 operable to indicate a depth of penetration of the device 1 in a patient, as for example, at a surgical site. In the embodiment shown in FIGS. 1,2, a depth scale 9 is provided on the shank 7 and preferably needs no moving indicator parts to provide at least part of the shank depth reading as measured from the distal end of the shank 7. In the embodiment of the invention shown in FIGS. 1,2, the scale 9 is applied to the outer surface 14 and uses a body portion such as a bone 18 to cooperate therewith to indicate depth of penetration by having an extremity portion, such as an outer surface 19 of the bone 18, align with a color zone segment 20A-G in the color zone 20 to indicate depth of penetration of the shank 7. The length of a color zone segment 20A, B, C, D, E, F, G along the longitudinal length of the color zone can be any suitable dimensions so long as it can be readily perceived by a user. Preferably, a color zone segment 20A-G extends around the entire perimeter of the shank 7. In the illustrated structure, the first few color zone segments and the last color zone segment, 20A-C, G are 10 mm long while the color zones 20D-F are 5 mm long as examples. It is to be understood that any desired lengths and number of color zones can be provided. The color displayed in one color zone segment 20A-G should be readily distinguishable from the immediately adjacent color zone(s). For example, color zone segment 20B may be a light blue while color zone segment 20A can be dark red and color zone segment 20C can be purple. Likewise, color zone segment 20G, an end color zone need only be distinguished from the color zone segment 20F and the color of the outer surface of the shank 7. The user will know by identifying the color in a color zone segment 20A-G that aligns with the surface of interest, like the surface 19, a depth of penetration and the user can interpolate within the color zone segment, a value within the range of depth values in that color zone segment. This structure requires no movable parts on the device 1 or operation step to align an indicator with a depth scale to indicate depth. The colors in the color zone segments 20A-G may be applied by anodizing since it will not chip off and allows for easy cleaning and sterilization.

FIG. 2 illustrates a second embodiment of the medical device 1. It is similar in construction to the device shown in FIG. 1, but has the rod 15 replaced with a wire 22 as is known in the art. One form of such a wire is referred to as a K-wire or Kirschner wire. The wire 22 would be typically used with the device 1 as a second step in the patient treatment procedure, after a hole is formed using the device 1 with the rod 15 (as shown in FIG. 1). The wire 22 can be used for an electro-physiological test to ensure there is no contact with a spinal nerve. A cannulated tap can also be deployed over the guide wire 22 to prepare the path for the pedicle screw. During tapping, the wire 22 can be tested continuously in real time to ensure the pedicle wall is not breached and there is no proximity to a spinal nerve. The wire 22 includes at least one color coded depth of penetration scale 9 such as color zone 24 and preferably includes a second color zone 25 with the color zone 24 being at the distal end portion 27 and the color zone 25 being adjacent the proximal end portion 28 of the wire 22. The color zones 24, 25 can be colored similar to the color zone 20 for convenience, providing color zone segments 24A-G and 25A-G. The color zones 24, 25 may be the same and thus be reversible in use if desired.

The color zone 25 can be used to indicate depth of penetration of the distal end portion 27 of 22 wire beyond the distal end portion 11 of the shank 7. The depth may be found by indexing a color zone segment 25A-G to a portion of the device 1, like a top portion of the handle 5 if still attached or a portion of the shank 7. The depth of penetration of the wire 22 is then the depth of penetration of the shank 7 plus the extension of the distal portion 27 of the wire 22 as indicated by an indexed color zone segment 25A-G. The depth of penetration of the wire 22 is also indicated by the color zone 24 when the shank 7 is removed by indexing a color zone segment 24A-G relative to a reference such as the outer surface 19. This second indexing of the wire 22 can be used to verify depth of penetration or instruct a user to modify depth of penetration of the wire 22.

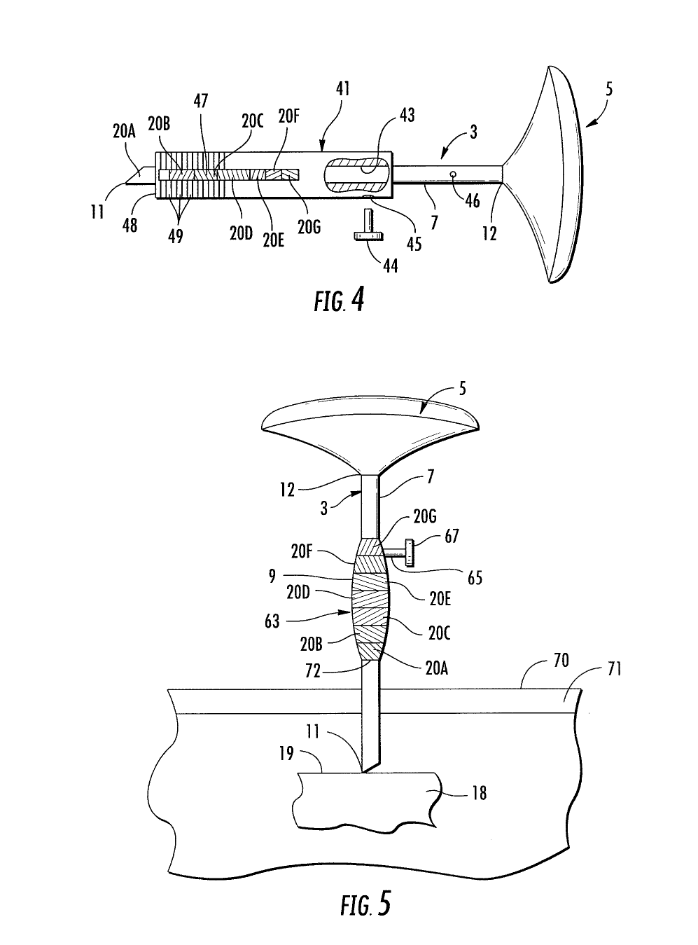

FIG. 3 illustrates a conversion device 35 useful in the operation of the medical device of the present invention such as the described device 1. The conversion device 35 is a reference material in the form of a conversion chart. It can be in the form of a printed material provided with the medical device 1 and provide information for a user to convert the color zones into depth of penetration of the device 1 and wire 22 by coordinating a color zone segment color to depth of penetration values. It can be provided as a separate device or may be provided on the shank 7 or wire 22 if there is enough space to provide the information. As shown, the conversion device 35 is auxiliary to the probe 3 and handle 5 and can be printed material. The conversion device 35 has a row of printed color indicia 37 with each color zone 20A-G being represented. Each color indicia 37A-G corresponds to a respective color zone segment 20A-G, 24A-G and 25A-G respectively and displays the same color. Depth indicia 39 is provided, and as shown, each depth indicia range 39A-G corresponds to a respective color indicia 20A-G, 24A-G, 25A-G and 37A-G to allow conversion of color to depth.

FIG. 4 shows an attachment scale device, designated generally 41, for use with the probe portion 3 to assist in refining depth of penetration measurements where needed. It provides for the ability of a user to more precisely determine the depth of penetration of the probe 3 by interpolating where in a color zone 20A-G, or one of the other color zones 24, 25, the depth indicating reference is located. The attachment scale device 41 is constructed to associate with the probe 3 and could also be used with a wire 22. Its use will be described as being associated with the probe 3 for convenience, but its use with a wire 22 would be similar. The attachment scale device 41 is constructed to mount to the probe 3, preferably in a removable manner. The attachment scale device 41 is also mounted to the probe 3 so as be axially movable relative to the probe in a selective manner. In the illustrated structure, the attachment scale device 41 is in the form of a tube having a longitudinal through bore 43 adapted to receive the probe 3 therethrough. A retainer 44 may be provided to fix the position of the attachment scale device 41 on the probe 3 and to configure them as an assembly to ensure they are together prior to surgery and to releasably retain the attachment scale device 41 in an out of the way position until needed for depth measurement value refinement. The retainer 44 can be a threaded pin that can be inserted through a hole 45 through the sidewall of the device 41 and into a threaded hole 46 in the probe 3. Other configurations could be used to effect a releasable retention. For example, the hole 45 could be threaded and the retainer 44 could then be used as a set screw.

The attachment scale device 41 is shown as being in the form of a tube with a through bore 43. It is provided with a window 47 extending along a portion of the longitudinal length of the attachment scale device 41 and opens into the bore 43 so that the exterior of the probe 3 can be viewed to see at least a portion of the color zone 20. The window 47 extends from adjacent an end 48 that is positioned more adjacent the distal end portion 11 of the shank 7 to be in overlying relation to at least one of the color zone segments 20A-G for viewing of one or more of the color segments 20A-G of the color zone 20 therethrough. Measurement indicia 49 are positioned adjacent the window 47, preferably along both sides of the window and are spaced apart longitudinally of the window 47. The spacing of the indicia 49 is determined by the desired resolution of scale. If the desired resolution is 2 mm, then the spacing can be 2 mm. In use, the device 1 is inserted into a patient and the shank 7 is inserted an approximate desired depth as indicated by a color zone segment 20A-G as desired and described herein. The scale device 41 can then be released from retention if not already released. The end 48 is then moved to the reference line such as the outer surface 19 of bone 18. The operator may then determine, more accurately, how far into a color zone 20 the reference line, e.g., the surface 19, is located by subtraction of the distance value of the specific reference indicia line 49 that most closely matches the proximal end of the color zone segment 20A-G. By way of example, if the reference surface 19 is located in the color zone segment 20G, it is known that the proximal end of that zone segment represents a penetration depth of 30 mm. The user then moves the end 48 into contact with the surface 19 and simply counts the number of indicia lines 49 from that proximal end and multiplies that number times the spacing and then subtracts that value from 30 mm to provide the penetration depth of the shank 7.

The invention is better understood by a description of its method of use. A probe device such as device 1 is selected that is suitably usable for the medical procedure to be performed on an animal. The animal can be any type of animal, including humans of any age, equine, bovine, swine, dogs, cats and the like. The probe 3 used has the above described plurality of different color zones along at least a portion of the length of the shank 7 of the device.

The shank 7 is inserted into an animal to a desired depth and the color zone indicating depth is noted by the user of the device. The user can identify the noted color on the reference material 35 and convert the color to depth of penetration of the shank 7. The color and/or depth of penetration can be selected prior to or after inserting or removing the shank 7. After the hole is formed to the proper depth, the rod 15 can be removed and replaced with the wire 22 and its depth of penetration determined and verified. The medical procedure is performed, which can include such procedures as forming a hole in bone, measuring proximity to nerve, guiding another device such as a tap, etc. Such procedures include spinal surgery, orthopedic surgery, bone marrow extraction and the like. The device 1 can form a hole for use in securing a screw to the bone in such procedures. The device can be also be used to extract a sample or inject a material. The device can be used to form a hole starting at the skin or an opening can be formed surgically to expose an inner body part such as a bone, and then the medical device 1 can be used.

FIG. 5 illustrates another embodiment of the medical device of the present invention and is designated generally as 61. It utilizes a probe portion 3 with handle portion 5. The probe 3 includes a shank 7 as described above. As disclosed above for the device 1, the device 61 is also provided with a color coded depth scale 9 having a plurality of depth indicating color zone segments 20A-G positioned along the length of the shank 7 thereof with adjacent color zone segments having different colors as described above. Each color of the color zone segments represents a different distance range from the distal end 11. In this embodiment, the depth scale is provided on a scale member 63 which is movably mounted to the shank 7 as by having the shank extend through a central through bore. The scale member 63 can be selectively fixed against longitudinal movement along the shank 7 as with a set screw 65 or the like, preferably being constructed to be loosened or tightened with a user's fingers. Such a screw 65 could be provided with a T shaped head 67 to allow finger tightening and loosening. The scale member 63 is provided with a color zone 20 having color zone segments 20A-G as described above. It provides the ability to determine depth of penetration without having to use a reference located at the body part of surgical interest, like the surface 19 of the bone 18. In use, the shank 7 is inserted until it reaches the body part of interest which may be hidden from view. In this example, the part of interest is a bone some distance in from an outer surface 70 of the body, for example, the skin 71. After the distal end 11 reaches the object of interest, the scale member 63 is released, if not already released, and is moved into contact with the reference surface 70 to be used, for example the outer surface of the skin 71. The scale member 63 is locked against relative movement on the shank 7 and into position on the shank 7 with the set screw 65. The device 61 is then used as the device 1 is used with the depth of penetration into the site of interest being indicated by the scale member 63 by referencing the reference surface 70 in a color zone segment 20A-G, as described above. The body part, in the described example the skin and underlying tissue such as muscle, is deformable to allow the scale member 63 to pass through preferably without having to make a larger incision. The distal end 72 of the scale member 63 may be contoured to provide for ease of penetration into the body part of interest like the skin 71. During use of the device 61, once the member 63 is locked at the proper position, no parts need be moved relative to the probe 3 to effect measurement of penetration depth of the shank 7.

The devices 1, 61 are particularly adapted for use in minimally invasive surgical procedures.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A medical tool with a depth indicating structure, the tool comprising:
a probe portion with a shank portion having a first distal end portion and a first proximal end portion, said shank portion having a longitudinally extending outer surface, said shank portion having a plurality of depth indicating first zone segments exposed along at least a portion of the length thereof with adjacent said first zone segments having different colors, each said first zone segment color representing a different distance range from the distal end portion; and
a handle member associated with the probe portion adjacent the proximal end portion.

2. The medical tool of claim 1 wherein the shank is tapered along at least a portion of the length between the first proximal and first distal end portions.

3. The medical tool of claim 1 wherein the shank has a generally cylindrical portion extending from adjacent the first distal end portion toward the first proximal end portion.

4. The medical tool of claim 1 wherein the first distal end portion is pointed.

5. The medical tool of claim 4 wherein the pointed end is beveled and the shank includes a through bore opening at the bevel.

6. The medical tool of claim 5 wherein the handle is removably mounted to the probe portion to selectively expose an open end of the bore adjacent the first proximal end portion.

7. The medical tool of claim 5 wherein the handle has at least a portion of thereof removable from association with the probe portion to selectively expose an open end of the bore.

8. The medical tool of claim 7 including a wire extending through the bore.

9. The medical tool of claim 8 wherein the wire has a second distal end portion, a second proximal end portion and a plurality of depth indicating second zone segments along a portion of the length thereof with adjacent said second zone segments having different colors, each said second zone segment color representing a different distance range from the second distal end portion.

10. The medical tool of claim 9 wherein the wire has a plurality of depth indicating third zone segments along a portion of the length thereof with adjacent third zone segments having different colors, each said third zone segment color representing a different distance range from the second distal end portion.

11. The medical tool of claim 7 including a rod positioned in the bore.

12. The medical tool of claim 7 wherein the medical tool is a jam-shidi needle.

13. The medical tool of claim 1 wherein the medical tool is a bone probe.

14. The medical tool of claim 1 including a reference material converting color of a first zone segment color to a depth of penetration range.

15. The medical tool of claim 10 including a reference material converting color of a first, second and third zone segment colors to a depth of penetration range.

16. The medical tool of claim 1 characterized by an absence of a movable depth indicator associated with the depth indicating zones.

17. The medical tool of claim 1 including a scale device movably mounted on the probe portion and being selectively positionable in overlying relation to at least one of the first zone segments, said scale device having a plurality of dimension indicia thereon adjacent the window.

18. The medical tool of claim 17 wherein the scale device has a window positioned for overlying at least one of the first zone segments with the dimension indicia being positioned adjacent the window.

19. The medical tool of claim 17 wherein at least some of the dimension indicia is spaced closer together than the width of at least one of the first zone segments.

20. The medical tool of claim 1 wherein at least one of the first zone segments is on an outer surface of the shank.

21. The medical tool of claim 1 including a scale member selectively movably mounted on the shank and at least one of the first zone segments being on an outer surface of the scale member.

22. The medical tool of claim 21 including a retainer associated with the scale member and operable to releasably fix the scale member at a selected axial position on the shank.

23. A method of determining depth of penetration of a medical tool into a portion of a patient, said method including:
selecting a probe usable for the medical procedure to be performed, said probe having a shank with a plurality of different color first zone segments along at least a portion of the length of the shank;
inserting the shank into an animal; and
determining depth of penetration of the shank by referencing a said color of a first zone segment to a portion of a body part of the animal.

24. The method of claim 23 wherein the referenced color of a first zone segment is compared to a color in a reference material and coordinates the reference color in a first zone segment to a depth of penetration of the shank.

25. The method of claim 23 wherein the color indicating depth of penetration values is selected prior to inserting the shank.

26. The method of claim 23 wherein the color indicating depth of penetration is coordinated to depth of penetration values after inserting of the shank.

27. The method of claim 23 including removing the shank from the animal, and wherein the color indicating depth of penetration is coordinated to depth of penetration values after removing the shank.

28. The method of claim 24 wherein the reference material is auxiliary to the medical tool.

29. The method of claim 24 wherein the reference material is on the medical tool.

30. The method of claim 24 including:
inserting a wire through the shank, the wire having a plurality of different color second zone segments along at least a portion of the length of the wire;
inserting the wire into the animal; and
determining depth of penetration of the wire into a portion of a body part of the animal by referencing a said color of a second zone segment to at least one portion of a body part of the animal and the probe.

31. The method of claim 30 wherein the wire has at least two sets of said different color second zone segments and determines depth of penetration to both a body part of the animal and the probe.

32. The method of claim 23 wherein at least some of the first zone segments are displayed on a scale member and the scale member is moved to a reference position along the shank and secured in place after an initial penetration of a body part of the animal.

33. A medical tool with a depth indicating structure, the tool comprising:
a wire having opposite first and second ends and an intermediate portion extending therebetween, said wire having a longitudinally extending outer surface with a plurality of depth indicating first zone segments exposed along at least a portion of the length of the wire from adjacent said first end with adjacent said first zone segments having different colors, each said first zone segment color representing a different distance range from said first end.

34. The medical tool of claim 33 including a plurality of depth indicating second zone segments exposed along at least a portion of the length of the wire from adjacent said second end with adjacent said second zone segments having different colors, each said second zone segment color representing a different distance range from said second end.

35. The medical tool of claim 33 wherein said wire is a K-wire.
